(19) United States
(12) Patent Application Publication
(10) Pub. No.: US 2014/0338673 A1
(43) Pub. Date: Nov. 20, 2014

(54) Welded Cuff

(71) Applicant: Engineered Medical Systems Inc., Indianapolis, IN (US)

(72) Inventor: Brad H. Quinn, Indianapolis, IN (US)

(21) Appl. No.: 13/893,927

(22) Filed: May 14, 2013

(51) Int. Cl. A61M 16/04; B29C 65/04; B29C 65/00

(52) U.S. Cl. CPC A61M 16/0445; A61M 16/0465; B29C 66/02241; B29C 65/04; A61M 2207/00; A61M 2205/0216; B29L 2031/7548
USPC 128/207.15; 156/256

(57) ABSTRACT

A cuff for placement on the outside surface of a first tube, for example, a tracheostomy or endotracheal tube, and a method of making the cuff. A pattern of the cuff including first and second end regions by which the cuff is to be attached to the first tube is RF welded onto second tube of a thin pliable RF weldable polymer. Polymer material outside the pattern is removed, leaving the length of the cuff including the first and second end regions. The length of the cuff including the first and second end regions is turned inside out to place the cut edge of the weld of the RF welded seam on the inside. The first end region is slipped onto an end of the first tube. The first end region is turned inside out again to orient the cut edge of the weld of the RF welded seam away from the first tube. The first end region is attached to the first tube. The second region is slipped onto the end of the first tube and turned inside out again to orient the cut edge of the weld of the RF welded seam away from the first tube, and attached to the first tube.

### FIELD

This invention relates to methods of forming inflatable cuffs on the outside walls of tracheostomy cannulas, endotracheal tubes and other tubular devices.

### BACKGROUND

The following recently issued U.S. Patents and numerous others describe and/or illustrate cuffs of this general type: U.S. Pat. Nos. 8,375,952; 8,371,303; 8,366,612; RE43,886; 8,343,408; 8,313,687; 8,312,879; 8,308,787; 8,307,830; 8,307,824; 8,281,789; 8,280,489; and, 8,220,454. There are also the devices illustrated and described in U.S. Pat. Nos. 5,469,863, 6,526,977 and 7,987,851, Kimberly-Clark Microcuff Pediatric Endotracheal Tube, Kimberly-Clark Worldwide, 2006, and Phoenurse Polyurethane Female Condom, Tianjin CondomBao Medical Polyurethane Tech. Co., LTD, Tianjin, China. The disclosures of these references are hereby incorporated herein by reference. The above listing is not intended to be a representation that a complete search of all relevant art has been made, or that no more pertinent art than that listed exists, or that the listed art is material to patentability. Not should any such representation be inferred.

### SUMMARY

A method for making a cuff for placement on the outside surface of a first tube comprises: providing a second tube of a thin pliable bondable polymer; bonding a pattern of a cuff including first and second end regions by which the cuff is to be attached to the first tube on the second tube; cutting the material outside the pattern; turning the length inside out to place the cut edge of the bonded seam on the inside of the length cut from the pattern; moving the first end region onto an end of the first tube; turning the first end region inside out again to orient the cut edge of the bonded seam away from the first tube; attaching the first end region to the first tube; moving the second region onto the end of the first tube; turning the second end region inside out again to orient the cut edge of the bonded seam away from the first tube; and attaching the second end region to the first tube.

Illustratively, placement on the outside surface of a first tube comprises placement on the outside surface of one of a tracheostomy tube and an endotracheal tube.

Illustratively, providing a tube of polymer comprises providing a tube of polyurethane (PU), polyvinyl chloride (PVC), thermoplastic elastomer(s) (TPE) or other suitable material(s).

Illustratively, providing a second tube of a thin pliable bondable polymer comprises providing a second tube having a wall thickness in the range of about 5 μm to about 50 μm.

Illustratively, bonding a pattern comprises radio frequency (RF) welding a pattern with an RF welding die.

Further illustratively, after bonding a pattern on the second tube and before turning the length inside out to place the cut edge of the bonded seam on the inside of the length cut from the pattern, the second tube is reduced to lengths.

Illustratively, attaching the first end region to the first tube comprises attaching the first end region to the first tube using (a) suitable adhesive(s), solvent(s), heat and/or other bonding method(s).

A cuff for placement on the outside surface of a first tube is made by: providing a second tube of a thin pliable bondable polymer; bonding a pattern of a cuff including first and second end regions by which the cuff is to be attached to the first tube on the second tube; removing the material outside the pattern; cutting from the pattern a length corresponding to the length of the cuff including the first and second end regions; turning the length inside out to place the cut edge of the bonded seam on the inside of the length cut from the pattern; moving a first end region onto an end of the first tube; turning the first end region inside out again to orient the cut edge of the bonded seam away from the first tube; attaching the first end region to the first tube; moving the second end region onto the end of the first tube; turning the second region inside out again to orient the cut edge of the bonded seam away from the first tube; and attaching the second end region to the first tube.

Illustratively, placement on the outside surface of a first tube comprises placement on the outside surface of one of a tracheostomy tube and an endotracheal tube.

Illustratively, providing a second tube of polymer comprises providing a second tube of polyurethane (PU), polyvinyl chloride (PVC), thermoplastic elastomer(s) (TPE) or other suitable material(s).

Illustratively, providing a second tube of a thin pliable bondable polymer comprises providing a second tube having a wall thickness in the range of about 5 μm to about 50 μm.

Illustratively, bonding a pattern comprises radio frequency (RF) welding a pattern with an RF welding die.

Further illustratively, after bonding a pattern on the second tube and before turning the length inside out to place the cut edge of the bonded seam on the inside of the length cut from the pattern, the second tube is reduced to lengths.

Illustratively, attaching the first end region to the first tube comprises attaching the first end region to the first tube using (a) suitable adhesive(s), solvent(s), heat and/or other bonding method(s).

A method for making a cuff for placement on the outside surface of a tube comprises: providing at least one sheet of a thin pliable bondable polymer; bonding a pattern of a cuff including first and second end regions by which the cuff is to be attached to the first tube on the at least one sheet; removing the material outside the pattern; cutting from the pattern a length corresponding to the length of the cuff including the first and second end regions; turning the length inside out to place the cut edge of the bonded seam on the inside of the length cut from the pattern; moving a first end region onto an end of the tube; turning the first end region inside out again to orient the cut edge of the bonded seam away from the tube to provide a smooth surface for attachment at an appropriate location on the tube; attaching the first end region to the tube; moving the second end region onto the end of the tube and toward the first region; turning the second end region inside out again to orient the cut edge of the bonded seam away from the tube to provide a smooth surface for attachment at an appropriate location on the tube; and attaching the second end region to the tube.

Illustratively, placement on the outside surface of a tube comprises placement on the outside surface of one of a tracheostomy tube and an endotracheal tube.

Illustratively, providing at least one sheet of polymer comprises providing at least one sheet of polyurethane (PU), polyvinyl chloride (PVC), thermoplastic elastomer(s) (TPE) or other suitable material(s).

Illustratively, providing at least one sheet of a thin pliable bondable polymer comprises providing at least one sheet having a wall thickness in the range of about 5 μm to about 50 μm or so to provide substantial pliability and avoid creases as the finished cuff is inflated in the trachea of a wearer.

Illustratively, bonding a pattern comprises radio frequency (RF) welding a pattern with an RF welding die.

Further illustratively, after bonding a pattern on the at least one sheet and before turning the length inside out to place the cut edge of the bonded seam on the inside of the length cut from the pattern, the at least one sheet is reduced to lengths.

Illustratively, attaching the first end region to the tube comprises attaching the first end region to the tube using (a) suitable adhesive(s), solvent(s), heat and/or other bonding method(s).

A cuff for placement on the outside surface of a tube is made by: providing at least one sheet of a thin pliable bondable polymer; bonding a pattern of a cuff including first and second end regions by which the cuff is to be attached to the tube on the at least one sheet; removing the material outside the pattern; cutting from the pattern a length corresponding to the length of the cuff including the first and second end regions; turning the length inside out to place the cut edge of the bonded seam on the inside of the length cut from the pattern; moving a first end region onto an end of the tube; turning the first end region inside out again to orient the cut edge of the bonded seam away from the tube to provide a smooth surface for attachment at an appropriate location on the tube; attaching the first end region to the tube; moving the second end region onto the end of the tube and toward the first region; turning the second region inside out again to orient the cut edge of the bonded seam away from the tube to provide a smooth surface for attachment at an appropriate location on the tube; and attaching the second end region to the tube.

Illustratively, placement on the outside surface of a tube comprises placement on the outside surface of one of a tracheostomy tube and an endotracheal tube.

Illustratively, providing at least one sheet of polymer comprises providing at least one sheet of polyurethane (PU), polyvinyl chloride (PVC), thermoplastic elastomer(s) (TPE) or other suitable material(s).

Illustratively, providing at least one sheet of a thin pliable bondable polymer comprises providing at least one sheet having a wall thickness in the range of about 5 μm to about 50 μm or so to provide substantial pliability and avoid creases as the finished cuff is inflated in the trachea of a wearer.

Illustratively, bonding a pattern comprises radio frequency (RF) welding a pattern with an RF welding die.

Further illustratively, after bonding a pattern on the at least one sheet and before turning the length inside out to place the cut edge of the bonded seam on the inside of the length cut from the pattern, the at least one sheet is reduced to lengths.

Illustratively, attaching the first end region to the tube comprises attaching the first end region to the tube using (a) suitable adhesive(s), solvent(s), heat and/or other bonding methods(s).

### BRIEF DESCRIPTION OF THE DRAWINGS

The invention may best be understood by referring to the following detailed description and accompanying drawings which illustrate the invention. In the drawings:

FIGS. 1a-e illustrate steps in the production of a cuffed tube according to an embodiment of the invention;

FIG. 2a-f illustrate steps in the production of a cuff;

FIGS. 3a-e illustrate steps in the production of a cuffed tube according to an embodiment of the invention;

FIGS. 4a-d illustrate steps in the production of a cuff;

FIG. 5 illustrates a much-enlarged fragmentary cross section taken generally along lines 5-5 of FIGS. 1a and 3a;

FIG. 6 illustrates a much-enlarged fragmentary cross section taken generally along lines 6-6 of FIGS. 1e and 3e;

FIG. 7 illustrates a simplified, highly diagrammatic view of an RF welding apparatus.

### DETAILED DESCRIPTIONS OF ILLUSTRATIVE EMBODIMENTS

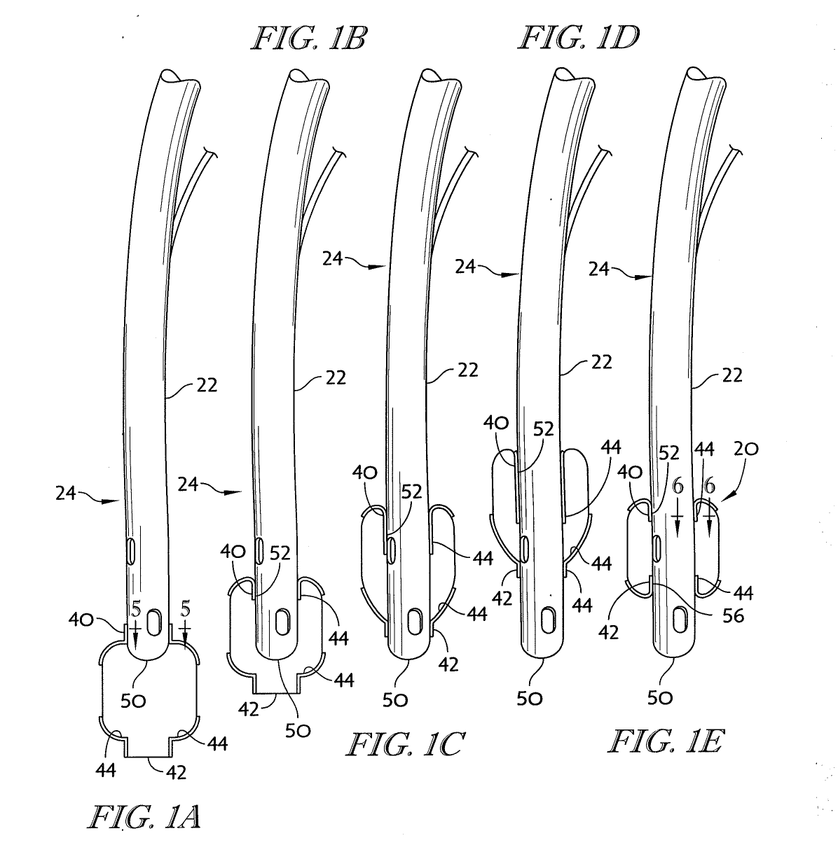

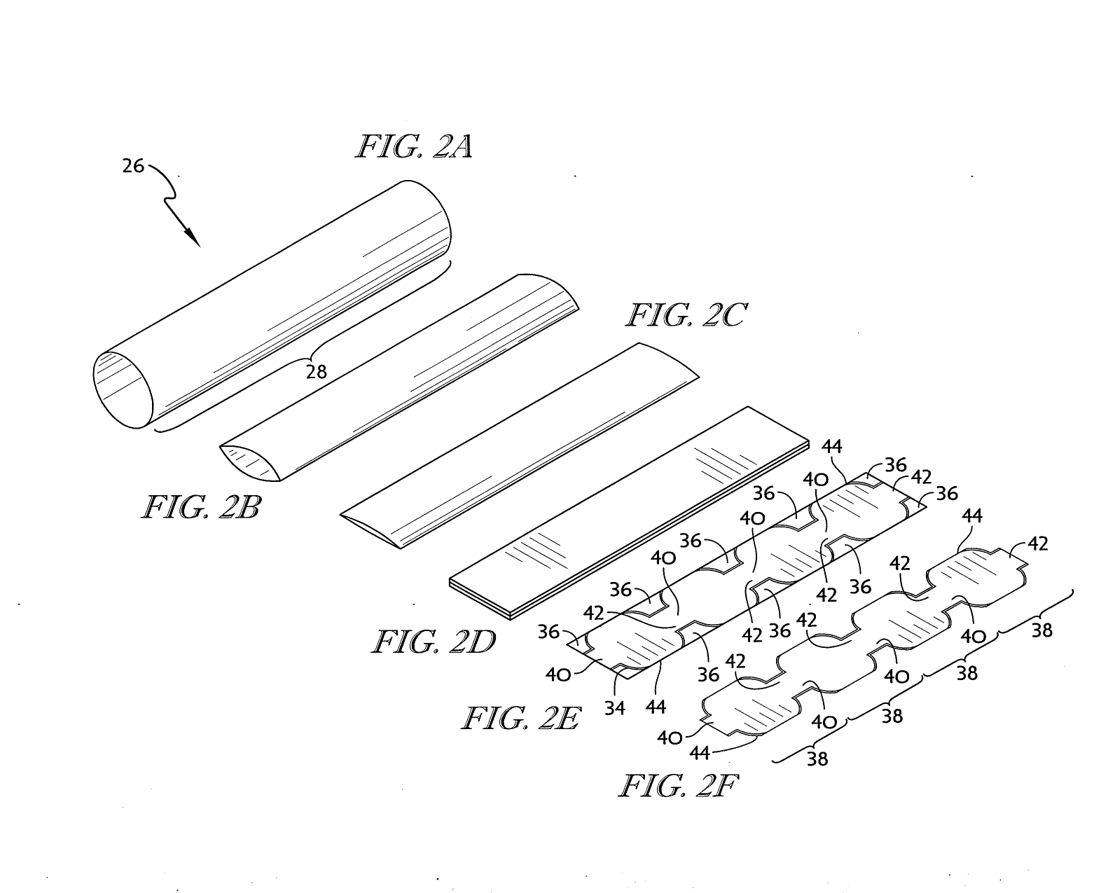

According to an embodiment, an inflatable cuff 20, FIG. 1e, for placement on the outside surface 22 of a tracheostomy tube, endotracheal tube or the like 24 begins as a tube 26, FIG. 2a, of polyurethane (PU), polyvinyl chloride (PVC), thermoplastic elastomer(s) (TPE) or other suitable material(s) extruded, for example, on a mandrel, or blown from a melt. The wall thickness of the tube 26 is in the range of about 5 μm to about 50 μm or so to provide substantial pliability and avoid creases as the finished cuff 20 is inflated in the trachea of a wearer. Predetermined lengths 28 of the tube 26 are collapsed flat, FIGS. 2b-d. An RF welding die 32, FIG. 7, welds a pattern 34, FIGS. 2e-f, on the length 28 of the tube 26 and the material 36 outside the pattern 34 is removed, compare FIGS. 1e-f, and recycled or discarded. The pattern 34 is cut into lengths 38 corresponding to the length of the cuff 20 including the end regions 40, 42 by which the cuff 20 will be attached to, for example, an endotracheal tube 24.

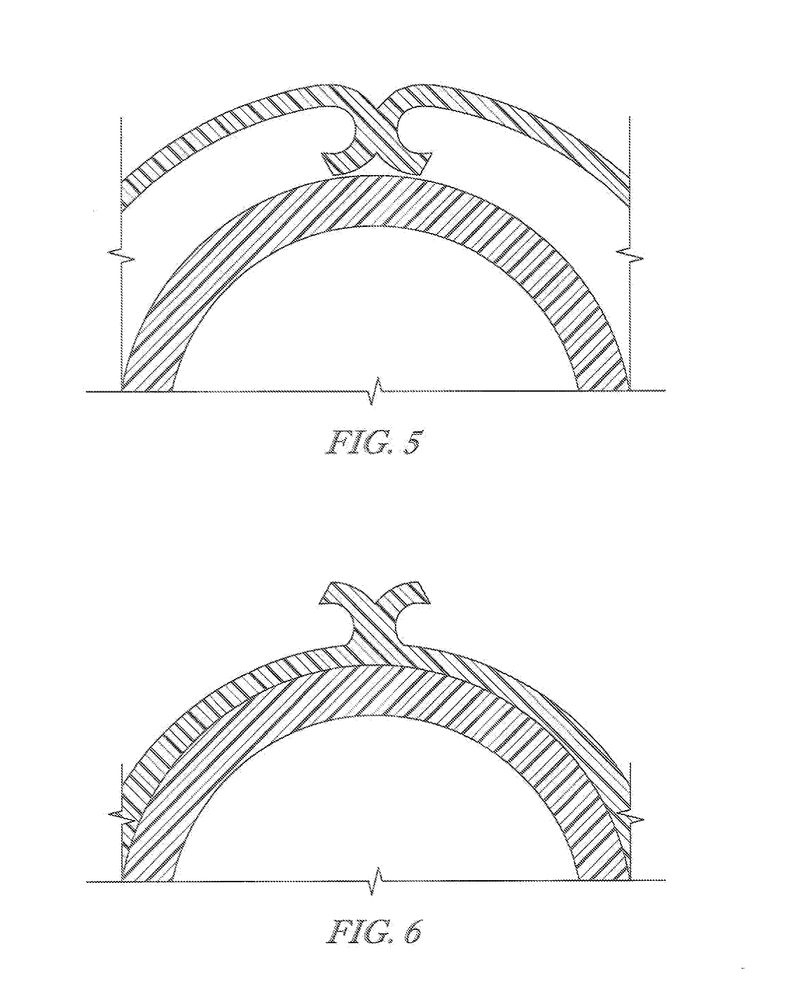

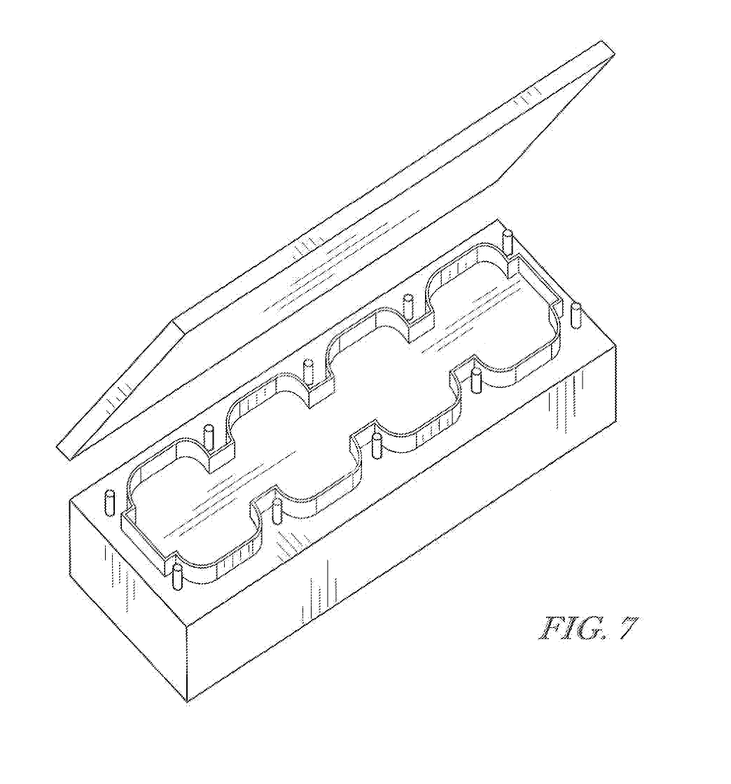

Each length 38 is turned inside out, FIG. 1a, placing the entire weld 44 of the RF welded seam 46, FIGS. 5-6, on the inside of the structure 38. A first end region 40 is pushed onto the end 50 of the tube 24, FIGS. 1a and 5, and first end region 40 is then turned inside out again. FIGS. 1b and 6, as first end region 40 is advanced, to provide a smooth surface 52. The second region 42 is slid onto the end 50 of the tube 24, FIGS. 1c and 5, pushed close to the first region 40, FIG. 1d, and then pulled away from the first region 40 while permitting the second region 42 to turn inside out again as well, FIGS. 1e and 6, so that its smooth surface 56 faces the surface 22 of the tube 24. Surfaces 52, 56 are sealed to the tube 24 in a fluid-tight manner using (a) suitable adhesive(s), solvent(s), heat and/or other bonding method(s).

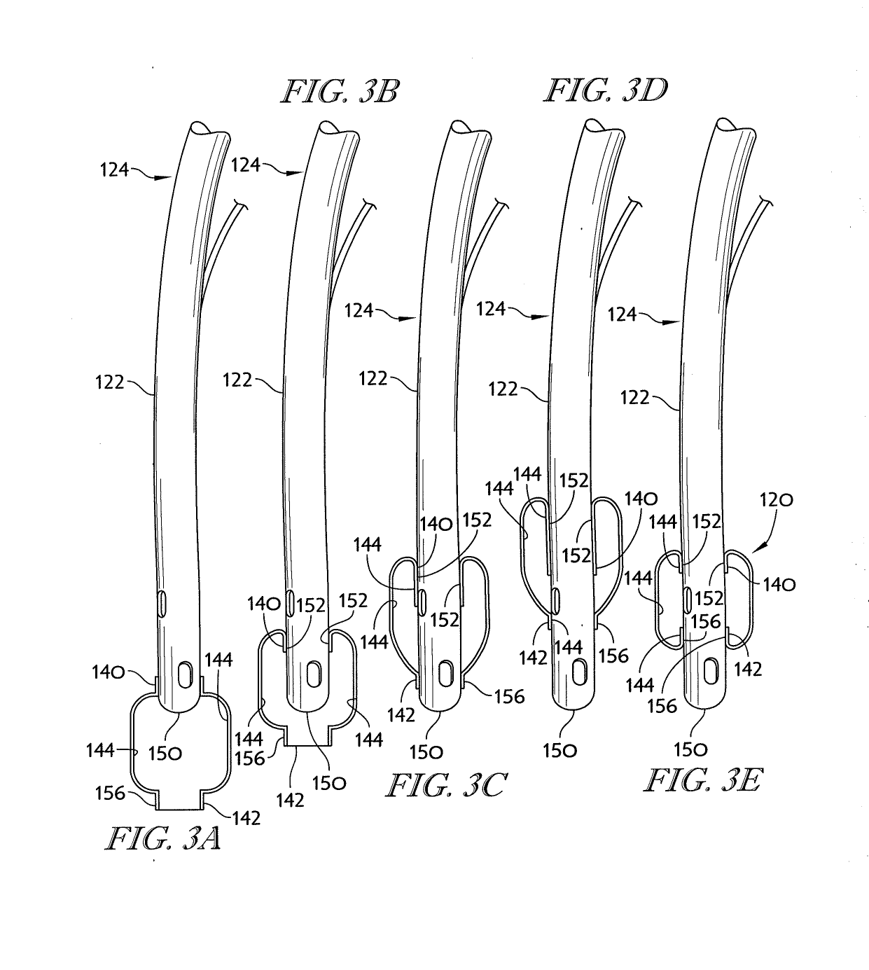

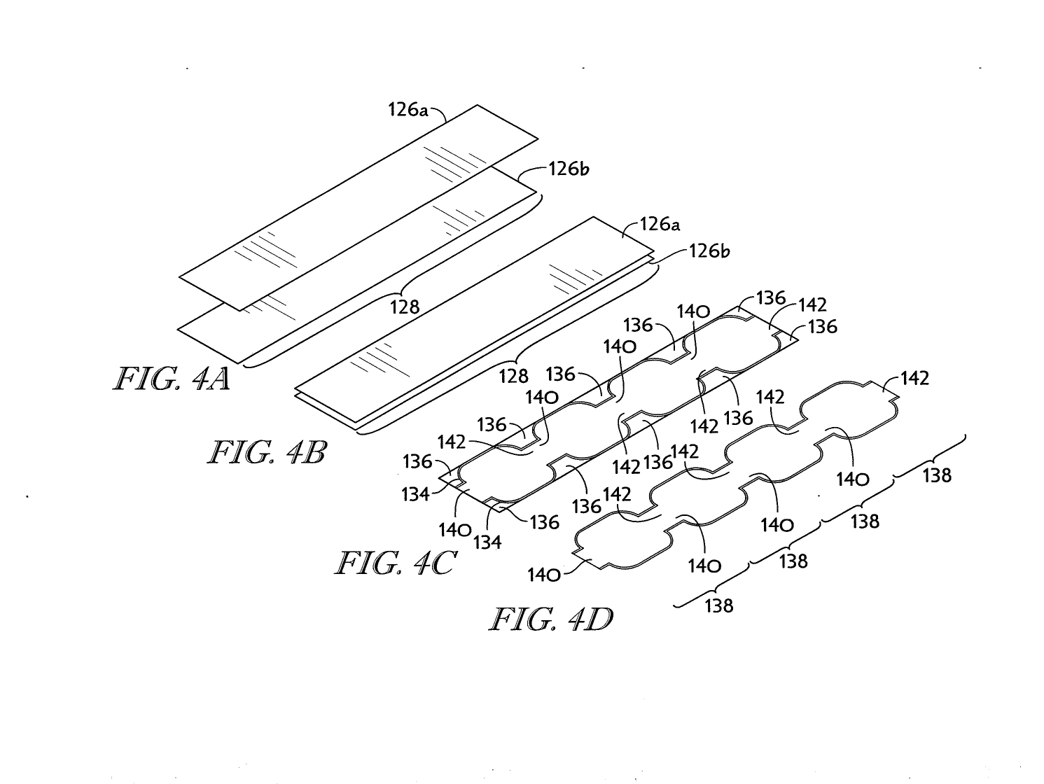

According to another embodiment, an inflatable cuff 120, FIG. 3e, for placement on the outside surface 122 of a tracheostomy tube, endotracheal tube or the like 124 begins as at least one folded sheet, or illustratively, two sheets or strips 126a, 126b, FIGS. 4a-b, of polyurethane (PU), polyvinyl chloride (PVC), thermoplastic elastomer(s) (TPE) or other suitable material(s) laid one on the other. The wall thicknesses of the two sheets or strips 126a, 126b again are in the range of about 5 μm to about 50 μm or so to provide substantial pliability and avoid creases as the finished cuff 120 is inflated in the trachea of a wearer. Predetermined lengths 128 of the two sheets or strips 126a, 126b are welded together using an RF welding die 132 that at the same time welds a pattern 134, FIG. 4c, on the length 128 of joined strips 126a, 126b. The material 136 outside the pattern 134 is removed, compare FIGS. 4c-d, and recycled or discarded. The pattern 134 is cut into lengths 138 corresponding to the length of the cuff 120 including the end regions 140, 142 by which the cuff 120 will be attached to, for example, an endotracheal tube 124.

Each length 138 is turned inside out, FIG. 3a, placing the entire weld 144 of the RF welded seam 146, FIGS. 5-6, on the inside of the structure 138. A first end region 140 is pushed onto the end 150 of the tube 124, FIGS. 3a and 5, and first end region 140 is then turned inside out again, FIGS. 3b and 6, as first end region 140 is advanced, to provide a smooth surface 152. The second region 142 is slid onto the end 150 of the tube 124, FIGS. 3c and 5, pushed close to the first region 140, FIG. 3d, and then pulled away from the first region 140 while permitting the second region 142 to turn inside out again as well, FIGS. 3e and 6, so that its smooth surface 156 faces the surface 122 of the tube 124. Surfaces 152, 156 are sealed to the tube 124 in a fluid-tight manner using (a) suitable adhesive(s), solvent(s), heat and/or other bonding method(s).

While polyurethane (PU), polyvinyl chloride (PVC), thermoplastic elastomer(s) (TPE) or other suitable material(s) is (are) the material(s) used in the above examples, any material which can be blown, extruded or otherwise fashioned into thin, highly pliable, biocompatible cuffs 20, 120 can be used. While RF welding has been described as the bonding method in the above examples, RF welding or other suitable bonding technique(s) can be used, as appropriate for the different materials which may be used for tube 26 or sheet(s) or strip(s) 126a, (126b).

What is claimed is:

1. A method for making a cuff for placement on the outside surface of a first tube, the method comprising providing a second tube of a thin pliable bondable polymer, bonding a pattern of a cuff including first and second end regions by which the cuff is to be attached to the first tube on the second tube, removing the material outside the pattern, turning the length inside out to place the cut edge of the bonded seam on the inside of the length cut from the pattern, moving the first end region onto an end of the first tube, turning the first end region inside out again to orient the cut edge of the bonded seam away from the first tube, attaching the first end region to the first tube, moving the second region onto the end of the first tube, turning the second end region inside out again to orient the cut edge of the bonded seam away from the first tube, and attaching the second end region to the first tube.

2. The method of claim 1 wherein placement on the outside surface of a first tube comprises placement on the outside surface of one of a tracheostomy tube and an endotracheal tube.

3. The method of claim 1 wherein providing a tube of polymer comprises providing a tube of polyurethane (PU), polyvinyl chloride (PVC), thermoplastic elastomer(s) (TPE) or other suitable material(s)

4. The method of claim 1 wherein providing a second tube of a thin pliable bondable polymer comprises providing a second tube having a wall thickness in the range of about 5 μm to about 50 μm.

5. The method of claim 1 wherein bonding a pattern comprises radio frequency (RF) welding a pattern with an RF welding die.

6. The method of claim 1 further comprising, after bonding a pattern on the second tube and before turning the length inside out to place the cut edge of the bonded seam on the inside of the length cut from the pattern, reducing the second tube to lengths.

7. The method of claim 1 wherein attaching the first end region to the first tube comprises attaching the first end region to the first tube using (a) suitable adhesive(s), solvent(s), heat and/or other bonding method(s).

8. A cuff for placement on the outside surface of a first tube, the cuff made by providing a second tube of a thin pliable bondable polymer, bonding a pattern of a cuff including first and second end regions by which the cuff is to be attached to the first tube on the second tube, removing the material outside the pattern, cutting from the pattern a length corresponding to the length of the cuff including the first and second end regions, turning the length inside out to place the cut edge of the bonded seam on the inside of the length cut from the pattern, moving a first end region onto an end of the first tube, turning the first end region inside out again to orient the cut edge of the bonded seam away from the first tube, attaching the first end region to the first tube, moving the second end region onto the end of the first tube, turning the second region inside out again to orient the cut edge of the bonded seam away from the first tube, and attaching the second end region to the first tube.

9. The cuff of claim 8 wherein placement on the outside surface of a first tube comprises placement on the outside surface of one of a tracheostomy tube and an endotracheal tube.

10. The cuff of claim 8 wherein providing a second tube of polymer comprises providing a second tube of polyurethane (PU), polyvinyl chloride (PVC), thermoplastic elastomer(s) (TPE) or other suitable material(s).

11. The cuff of claim 8 wherein providing a second tube of a thin pliable bondable polymer comprises providing a second tube having a wall thickness in the range of about 5 μm to about 50 μm.

12. The cuff of claim 8 wherein bonding a pattern comprises radio frequency (RF) welding a pattern with an RF welding die.

13. The cuff of claim 8 further comprising after bonding a pattern on the second tube and before turning the length inside out to place the cut edge of the bonded seam on the inside of the length cut from the pattern reducing the second tube to lengths.

14. The cuff of claim 8 wherein attaching the first end region to the first tube comprises attaching the first end region to the first tube using (a) suitable adhesive(s), solvent(s), heat and/or other bonding method(s).

15. A method for making a cuff for placement on the outside surface of a tube, the method comprising providing at least one sheet of a thin pliable bondable polymer, bonding a pattern of a cuff including first and second end regions by which the cuff is to be attached to the first tube on the at least one sheet, removing the material outside the pattern, cutting from the pattern a length corresponding to the length of the cuff including the first and second end regions, turning the length inside out to place the cut edge of the bonded seam on the inside of the length cut from the pattern, moving a first end region onto an end of the tube, turning the first end region inside out again to orient the cut edge of the bonded seam away from the tube to provide a smooth surface for attachment at an appropriate location on the tube, attaching the first end region to the tube, moving the second end region onto the end of the tube and toward the first region, turning the second end region inside out again to orient the cut edge of the bonded seam away from the tube to provide a smooth surface for attachment at an appropriate location on the tube, and attaching the second end region to the tube.

16. The method of claim 15 wherein placement on the outside surface of a tube comprises placement on the outside surface of one of a tracheostomy tube and an endotracheal tube.

17. The method of claim 15 wherein providing at least one sheet of polymer comprises providing at least one sheet of polyurethane (PU), polyvinyl chloride (PVC), thermoplastic elastomer(s) (TPE) or other suitable material(s).

18. The method of claim 15 wherein providing at least one sheet of a thin pliable bondable polymer comprises providing at least one sheet having a wall thickness in the range of about 5 μm to about 50 μm or so to provide substantial pliability and avoid creases as the finished cuff is inflated in the trachea of a wearer.

19. The method of claim 15 wherein bonding a pattern comprises radio frequency (RF) welding a pattern with an RF welding die.

20. The method of claim 15 further comprising after bonding a pattern on the at least one sheet and before turning the length inside out to place the cut edge of the bonded seam on the inside of the length cut from the pattern reducing the at least one sheet to lengths.

21. The method of claim 15 wherein attaching the first end region to the tube comprises attaching the first end region to the tube using (a) suitable adhesive(s), solvent(s), heat and/or other bonding method(s).

22. A cuff for placement on the outside surface of a tube, the cuff made by providing at least one sheet of a thin pliable bondable polymer, bonding a pattern of a cuff including first and second end regions by which the cuff is to be attached to the tube on the at least one sheet, removing the material outside the pattern, cutting from the pattern a length corresponding to the length of the cuff including the first and second end regions, turning the length inside out to place the cut edge of the bonded seam on the inside of the length cut from the pattern, moving a first end region onto an end of the tube, turning the first end region inside out again to orient the cut edge of the bonded seam away from the tube to provide a smooth surface for attachment at an appropriate location on the tube, attaching the first end region to the tube, moving the second end region onto the end of the tube and toward the first region, turning the second region inside out again to orient the cut edge of the bonded seam away from the tube to provide a smooth surface for attachment at an appropriate location on the tube, and attaching the second end region to the tube.

23. The cuff of claim 22 wherein placement on the outside surface of a tube comprises placement on the outside surface of one of a tracheostomy tube and an endotracheal tube.

24. The cuff of claim 22 wherein providing at least one sheet of polymer comprises providing at least one sheet of polyurethane (PU), polyvinyl chloride (PVC), thermoplastic elastomer(s) (TPE) or other suitable material(s).

25. The cuff of claim 22 wherein providing at least one sheet of a thin pliable bondable polymer comprises providing at least one sheet having a wall thickness in the range of about 5 μm to about 50 μm or so to provide substantial pliability and avoid creases as the finished cuff is inflated in the trachea of a wearer.

26. The cuff of claim 22 wherein bonding a pattern comprises radio frequency (RF) welding a pattern with an RF welding die.

27. The cuff of claim 22 further comprising after bonding a pattern on the at least one sheet and before turning the length inside out to place the cut edge of the bonded seam on the inside of the length cut from the pattern reducing the at least one sheet to lengths.

28. The cuff of claim 22 wherein attaching the first end region to the tube comprises attaching the first end region to the tube using (a) suitable adhesive(s), solvent(s), heat and/or other bonding method(s).
